(19) United States
(12) Patent Application Publication
(10) Pub. No.: US 2009/0078258 A1
(43) Pub. Date: Mar. 26, 2009

(54) Pressure regulation methods for positive pressure respiratory therapy

(76) Inventors: Bruce R. Bowman, Eden Prairie, MN (US); Vernon G. Menk, Minnetonka, MN (US)

Correspondence Address: CYR & ASSOCIATES, P.A., 605 U.S. Highway 169, Suite 300, Plymouth, MN, 55441, US

(21) Appl. No.: 11/903,477

(22) Filed: Sep. 21, 2007

(51) Int. Cl. A61M 16/06; A62B 7/00

(52) U.S. Cl. 128/204.26; 128/205.23

(57) ABSTRACT

Methods that may provide a positive pressure therapy to a user are described. The methods include detecting the user's breathing parameters and increasing the pressure delivered to the user from a sub-therapeutic pressure to a therapeutic pressure based upon the breathing parameters.

### BACKGROUND OF THE INVENTION

1. Field of the Invention

The present inventions relate to positive pressurized respiratory therapy and, more particularly, to apparatus and methods for managing pressure during positive pressure respiratory therapies.

2. Description of the Related Art

Positive airway pressure devices typically deliver pressurized air and/or other breathable gasses to the airways of a patient to prevent upper airway occlusion during sleep. The pressurized air is typically administered by a mask placed over the user's nose and/or mouth and at pressures ranging between about 4 cm to 20 cm of water. Positive airway pressure devices have become the devices of choice for the treatment of chronic sleep apnea and snoring. Many variations of positive airway pressure devices are now commercially available.

A typical positive airway pressure device includes a flow generator, a delivery tube and a mask. In various configurations, the mask may fit over the nose and, sometimes the mouth, may include nasal pieces that fit under the nose, may include nostril inserts into the nares, or some combination thereof. The masks frequently include one or more straps configured to secure the mask to the user so that pressurized air may be delivered from the flow generator for inhalation by the user.

For the comfort of the user, it may be beneficial to provide pressurized air to the user initially at a sub-therapeutic pressure and to increase the pressure to a therapeutic pressure over a period of time. Therefore, a need exists for positive airway pressure devices that may increase the pressure over time up to therapeutic pressures.

### SUMMARY OF THE INVENTION

Methods in accordance with the present inventions may resolve many of the needs and shortcomings discussed above and will provide additional improvements and advantages that may be recognized by those of ordinary skill in the art upon review of the present disclosure.

The methods may include initiating a pressure delivered to a user at a sub-therapeutic pressure. The methods may include sensing one or more breathing parameters. The methods may include calculating at least two pressure steps from the one or more breathing parameters, and the methods may include increasing the pressure by at least two pressure steps while the user is in a state of wakefulness.

Other features and advantages of the inventions will become apparent from the following detailed description and from the claims.

### BRIEF DESCRIPTION OF THE DRAWINGS

FIG. 1A illustrates a perspective view of an exemplary embodiment of a respiratory therapy apparatus in accordance with the present inventions;

FIG. 1B illustrates a perspective view of another exemplary embodiment of a respiratory therapy apparatus in accordance with the present inventions;

FIG. 2A illustrates diagrammatically an exemplary embodiment of the pressure delivered by the respiratory therapy apparatus;

FIG. 2B illustrates diagrammatically an exemplary embodiment of a breath;

FIG. 2C illustrates diagrammatically an exemplary embodiment of a pressure step;

FIG. 3A illustrates diagrammatically an exemplary embodiment of breathing parameters;

FIG. 3B illustrates diagrammatically an exemplary embodiment of pressure steps corresponding to the breathing parameters in FIG. 3A.

FIG. 4A illustrates diagrammatically an exemplary embodiment of breathing parameters;

FIG. 4B illustrates diagrammatically another exemplary embodiment of breathing parameters;

FIG. 4C illustrates diagrammatically an exemplary embodiment of pressure steps generally corresponding to the breathing parameters in FIGS. 4A and 4B.

FIG. 5A illustrates diagrammatically an exemplary embodiment of breathing parameters;

FIG. 5B illustrates diagrammatically another exemplary embodiment of breathing parameters;

FIG. 5C illustrates diagrammatically an exemplary embodiment of a pressure step generally corresponding to the breathing parameters in FIGS. 5A and 5B;

FIG. 6A illustrates diagrammatically an exemplary embodiment of breathing parameters;

FIG. 6B illustrates diagrammatically another exemplary embodiment of breathing parameters;

FIG. 6C illustrates diagrammatically an exemplary embodiment of a pressure step generally corresponding to the breathing parameters in FIGS. 6A and 6B;

FIG. 7A illustrates diagrammatically an exemplary embodiment of breathing parameters;

FIG. 7B illustrates diagrammatically another exemplary embodiment of breathing parameters;

FIG. 7C illustrates diagrammatically an exemplary embodiment of a pressure step generally corresponding to the breathing parameters in FIGS. 7A and 7B;

FIG. 8A illustrates a schematic diagram of an exemplary embodiment of portions of a respiratory therapy apparatus in accordance with the present inventions; and,

FIG. 8B illustrates a schematic diagram of an exemplary embodiment of portions of a respiratory therapy apparatus in accordance with the present inventions.

All Figures are illustrated for ease of explanation of the basic teachings of the present inventions only; the extensions of the Figures with respect to number, position, relationship and dimensions of the parts to form the embodiment will be explained or will be within the skill of the art after the following description has been read and understood. Further, the dimensions and dimensional proportions to conform to specific force, weight, strength, flow and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they generally correspond with airflow within an apparatus in accordance with the present inventions.

### DETAILED DESCRIPTION OF THE INVENTION

The respiratory therapy apparatus 10 may include a flow generator 20 and a user interface 40. In certain aspects, the respiratory therapy apparatus 10 may also include a delivery tube 30. The flow generator 20 is provided as a source of pressurized air. When present, the delivery tube 30 is configured to communicate pressurized air from the flow generator 20 to the user interface 40, which, in turn, is configured to communicate the pressurized air into the airways of a user. The user interface 40 may be configured to be secured relative to the user's head such that a positive pressure therapy may be administered to a user by the respiratory therapy apparatus 10 as the user sleeps. In some aspects, the respiratory therapy apparatus 10 may be configured to increase the pressure 106 of the pressurized air delivered to the user in one or more pressure steps 100 based upon user demand. In some aspects, the respiratory therapy apparatus 10 may be configured to detect the user's breathing parameters 88, and the user demand may be based upon the user's breathing parameters 88. In some aspects, the respiratory therapy apparatus 10 may be configured to detect the user's progress toward the sleep state 128 based upon the user's breathing parameters 88, and user demand may be based upon the user's progress toward the sleep state 128.

The Figures generally illustrate exemplary embodiments of respiratory therapy apparatus 10. These illustrated apparatus 10 and methods are not meant to limit the scope of coverage but, instead, to assist in understanding the context of the language used in this specification and in the appended claims. Accordingly, the appended claims may encompass variations that differ from the illustrations.

The respiratory therapy apparatus 10 may be configured to provide one or more positive airway pressure therapies to the user. The one or more positive airway pressure therapies may include continuous positive airway pressure therapy (CPAP), bi-level positive airway pressure therapy (BPAP), auto positive airway pressure therapy (auto-PAP), proportional positive airway pressure therapy (PPAP), and/or other positive airway pressure therapies as will be recognized by those of ordinary skill in the art upon review of this disclosure.

The respiratory therapy apparatus 10 typically includes a user interface 40. The user interface 40 is generally configured to communicate pressurized air communicated from the flow generator 20 into the airways of a user. The user interface 40 may be generally configured to be secured to a user and to communicate pressurized air into the airway of a user. The user interface 40 can include a mask 60 configured to be secured over the airways of a user. In certain aspect, the mask 60 may include a cap, one or more support bands 44, or other elements as will be recognized by those skilled in the art to secure the mask 60 to the user. The user interface 40 may define an interface passage 74. The interface passage 74 may be in fluid communication with a chamber 66 defined by the mask 60 to communicate pressurized air through the interface passage 74. The user interface 40 may also or alternatively include a mount 48 and various other features such as pads that allow the user interface 40 including the mask 60 to be affixed to the user and that maintain a proper orientation of the user interface 40 including the mask 60 with respect to the user.

The mask 60 portion of the user interface 40 may be configured to communicate the pressurized air generated by the flow generator 20 to the user's airways. In various aspects, the mask 60 may be positioned about the user's nose, the user's mouth, or both the user's nose and mouth in order to provide a generally sealed connection to the user for the delivery of pressurized air for inhalation. A pressure 106 greater than atmospheric pressure may be provided within the sealed connection. Accordingly, portions of the mask 60 may be formed of soft silicone rubber or similar material that may provide a seal and that may also be generally comfortable when positioned against the user's skin. In various aspects, the mask 60 may include nasal pieces that fit under the user's nose, nostril inserts into the user's nares, or some combination thereof.

The flow generator 20 may include a flow generator housing 22 defining an outlet 24, with the flow generator 20 adapted to deliver pressurized air to the outlet 24. In order to deliver pressurized air to the outlet 24, the flow generator 20 may include one or more of various motors, fans, pumps, turbines, ducts, inlets, conduits, passages, mufflers, and other components, as will be recognized by those of ordinary skill in the art upon review of the present disclosure.

In some aspects, the flow generator 20 may be included in the user interface 40 such that the flow generator 20 is generally secured about the user's head. The outlet 24 of the flow generator 20 may fluidly communicate with the interface passage 74 to convey pressurized air to the user for inhalation.

In other aspects, the flow generator 20 is separated from the user interface 40. A delivery tube 30 may then be secured to an outlet 24 of the flow generator 20 to convey pressurized air from the flow generator 20 to the interface passage 74 defined by the user interface 40. In one aspect, the delivery tube 30 may be configured as an elongated flexible tube. The delivery tube 30 may be composed of a lightweight plastic, and often has a ribbed configuration. A delivery tube passage 36 defined by the delivery tube 30 may extend between a proximal end 32 and a distal end 34 of the delivery tube 30. The proximal end 32 of the delivery tube 30 may be adapted to be secured to the flow generator 20 with the delivery tube passage 36 in fluid communication with the outlet 24 of the flow generator 20. The interface passage 74 defined by the user interface 40 may be secured to the distal end 34 of the delivery tube 30 to be in fluid communication with the delivery tube passage 36. Accordingly, pressurized air from the flow generator 20 may be communicated through the delivery tube passage 36 through the interface passage 74 and delivered to the user interface 40 for inhalation.

A control unit 26 may be included in the respiratory therapy apparatus 10 to control the respiratory therapy apparatus 10 including controlling the pressure of the pressurized air delivered to the user in order to deliver one or more positive airway pressure therapies to the user. The control unit 26 can be positioned within and/or on the flow generator housing 22, but may be otherwise positioned or located, including remotely, as will be recognized by those of ordinary skill in the art upon review of the present disclosure. In some aspects, portions of the control unit 26 may be located remotely. The control unit 26 may include one or more circuits and/or may include one or more microprocessors as well as computer readable memory. The control unit 26 may include various communication channels configured so that the control unit 26 may receive signals 212 from and output control signals 214 to various components of the respiratory therapy apparatus 10. Communication channels may include wire, fiberoptic, and various wireless technologies.

The control unit 26 may be adapted to control the respiratory therapy apparatus 10 in response to signals 212 indicative of the user's breathing parameters 88 received from one or more sensors 210 disposed about the respiratory therapy apparatus 10. The control unit 26 may be configured to output one or more control signals 214 to various components of the flow generator 20 and other components of the respiratory therapy apparatus 10 and otherwise adapted to control the respiratory therapy apparatus 10 in response to the signals 212 from the one or more sensors 210 in ways that would be recognized by those of ordinary skill in the art upon review of this disclosure.

In particular, the control unit 26 may be adapted to control the pressure 106 of the pressurized air delivered to the user by the respiratory therapy apparatus 10 in response to one or more signals 212 from the one or more sensors 210. In some exemplary aspects, the control unit 26 may control the pressure 106 delivered to the user in response to the one or more signals 212 by modulating the speed of a motor 220 that drives a fan or other air compressive device in the flow generator 20. In other exemplary aspects, the control unit 26 may modulate one or more valves 230 including other flow control devices disposed in the flow generator 20 or otherwise disposed throughout the respiratory therapy apparatus 10 in order to control the pressure of the pressurized air delivered to the user. In other exemplary aspects, the control unit 26 may control the pressure 106 delivered to the user in response to the one or more signals 212 by modulating both the speed of a motor 220 that drives a fan or other air compressive device in the flow generator 20 and one or more valves 230 including other flow control devices disposed in the flow generator 20 or otherwise disposed throughout the respiratory therapy apparatus 10 in order to control the pressure of the pressurized air delivered to the user.

The respiratory therapy apparatus 10, as directed by the control unit 26, may deliver pressurized air to the user at a sub-therapeutic pressure p_{B} and at a therapeutic pressure p_{T}, and may also deliver pressurized air to the user at one or more pressures 106 intermediate of the sub-therapeutic pressure p_{B} and the therapeutic pressure p_{T}. The sub-therapeutic pressure p_{B} is a non-therapeutic pressure typically provided at start-up of the respiratory therapy apparatus 10 as the user goes to bed. The sub-therapeutic pressure p_{B} may be initiated before, during, or after the user interface 40 is secured over the user's airways. The sub-therapeutic pressure p_{B} is typically a low pressure that the user finds comfortable at the start of respiratory therapy. In various aspects, this sub-therapeutic pressure p_{B} and corresponding airflow may serve to provide some initial support to the user's airway. The sub-therapeutic pressure p_{B} may be at least a pressure required to flush exhaled CO₂ out of the mask 60. A typical sub-therapeutic pressure may range from about 4 cm to about 6 cm of H₂O but could be greater for some individuals

The therapeutic pressure p_{T} may be a prescribed pressure established by a health care professional based upon the user's anatomy and physiology, and may be chosen as the minimum pressure required for support of the user's airways in order to prevent apneic events. The therapeutic pressure p_{T} is usually greater than the sub-therapeutic pressure p_{B}. The therapeutic pressure p_{T} may range typically from about 6 cm of H₂O to about 16 cm of H₂O, although for some individuals, the therapeutic pressure p_{T} may be about 20 cm of H₂O or more. While the therapeutic pressure p_{T} and the sub-therapeutic pressure p_{B} may vary from individual to individual, the p_{T} may be generally at least about 2 cm of H₂O above the sub-therapeutic pressure p_{B}.

It should be recognized that both the sub-therapeutic pressure p_{B} and the therapeutic pressure p_{T} may have, in various aspects, multiple pressure components, and the respiratory therapy apparatus 10 may adjust between these pressure components in various ways. For example, the therapeutic pressure p_{T} may include a pressure component generally delivered to the user during inhalation 95 and a pressure component generally delivered to the user during exhalation 99. Similarly, in various aspects, the sub-therapeutic pressure p_{B} may include a pressure component generally delivered to the user during inhalation 95 and a pressure component generally delivered to the user during exhalation 99. Other pressures 106 delivered to the user by the respiratory therapy apparatus 10 including pressures 106 intermediate the sub-therapeutic pressure p_{B} and the therapeutic pressure p_{T} may also include multiple pressure components, for example, a pressure component generally delivered to the user during inhalation 95 and a pressure component generally delivered to the user during exhalation 99.

In the respiratory therapy apparatus 10, the control unit 26 may receive one or more signals 212 from one or more sensors 210. The signals 212 may be indicative of the user's breathing parameters 88. The control unit 26 may be adapted to control the pressure 106 delivered to the user by the respiratory therapy apparatus 10 based upon the one or more signals 212. The control unit 26 may be configured to increase the pressure 106 delivered to the user in one or more pressure steps 100 from the sub-therapeutic pressure p_{B} to the therapeutic pressure p_{T} and to determine the one or more pressure steps 100 based upon the one or more signals 212.

As used herein, a breath 90 may begin with inhalation initiation 91 at the start of an inhalation 95 and include inhalation 95. The breath 90 may further proceed to exhalation initiation 97 and includes exhalation 99 to the completion of exhalation 99 and the inhalation initiation 91 of the succeeding breath 90. Alternatively, the breath 90 may be defined, for example, exhalation initiation 97 to the succeeding exhalation initiation 97, from inhalation peak 96 to the succeeding inhalation peak 96, from exhalation peak 98 to the succeeding exhalation peak 98, or in other ways recognized by those of ordinary skill in the art upon review of this disclosure. The breath 90 occurs over a corresponding breath interval 92, the breath interval 92 being the time required for the breath 90.

The breathing parameters 88 may include the breath interval 92 of the user and/or other features of the breath 90 indicative of the user's breath interval 92. The breathing rate, which is the inverse of the breath interval 92, may also be used as a breathing parameter 88. The breathing parameters 88 may include the breath airflow rate 116 and may include the breath airflow amplitude 94 and the mean breath airflow amplitude 132. The inhalation peak 96 may be defined as the maximum airflow rate into the user's air passages during inhalation 95 and the exhalation peak 98 may be defined as the maximum airflow rate expelled from the user's airway passages during exhalation 99. Breath airflow amplitude 94 may be measured, inter alia, as the amplitude of the inhalation peak 96, the amplitude of the exhalation peak 98, the difference between the inhalation peak 96 and the exhalation peak 98, or the root mean square of the difference between the inhalation peak 96 and the exhalation peak 98. The mean breath airflow amplitude 132 is the mean of the breath airflow rate 116 during inhalation 95.

The breathing parameters 88 may also include the breath volume 118 and may include the tidal volume 93 or minute ventilation, which is the sum of the tidal volume 93 over a minute. In addition, the breathing parameters 88 may include integral measures, measures of wave shapes, various rates of change, and statistical measures such as averages and moving averages, alone or in combination as will be recognized by those of ordinary skill in the art upon review of the present disclosure. The breathing parameters 88 may include various features of the breath 90 or series of breaths 90 as will be recognized by those of ordinary skill in the art upon review of the present disclosure.

In various aspects, the breathing parameters 88 may be indicative of the user's progress toward the sleep state 128 and/or attainment of the sleep state 128. The breathing pattern during the sleep state 128 is typically more rapid and shallow than during wakefulness 124. The tidal volume 93 while progressing toward the sleep state is typically reduced in comparison to the tidal volume during wakefulness 124, so that the minute ventilation is correspondingly reduced during the sleep state 128 in comparison to wakefulness 124. The mean breath airflow amplitude 132 is also typically reduced during the sleep state 128 in comparison to wakefulness 124. Breathing parameters 88 may include, in various aspects, other features of the user's breath 90 and/or breaths 90 indicative of the user's progress toward the sleep state 128 and/or attainment of the sleep state 128 as would be recognized by those of ordinary skill in the art upon review of this disclosure.

The sensors 210 may be configured to detect the breathing parameters 88 and to generate signals 212 indicative of the breathing parameters 88. The breathing parameters 88 may be detected, in various aspects, by measuring the airflow delivered to the patient by sensors 210 such as, for example, a pneumotach or a Pitot tube and determining inhalation 95 and exhalation 99 based on the direction of airflow. Other methods of detecting the breathing parameters 88 include use of sensors 210 such as a thermistor or thermocouple for measuring a temperature difference between air delivered to the user during inhalation 95 and air exhaled by the user during exhalation 99. Sensors 210 configured as pressure transducers may be employed in various aspects to detect breathing parameters 88. The sensors 210 could, in some aspects, include one or more components of the respiratory therapy apparatus 10 capable of generating signals indicative of the operation of the respiratory therapy apparatus 10. For example, the sensor 210 could detect breathing parameters 88 by detecting the electrical load on the motor 220, or current or power delivered to the motor in the flow generator 20. As another example, the sensor 210 could detect breathing parameters 88 by detecting the rotational rate (RPM) of the motor 220 particularly when the respiratory therapy apparatus 10 is operating in a pressure controlled feedback loop that maintains constant pressure during inhalation and exhalation loading by changing rotational rate (RPM). The electrical load and/or the rotational rate of the motor 220 may be correlated to the user's breathing parameters 88 such as the user's inhalation 95 and exhalation 99. Other sensors 210 may be utilized to detect the breathing parameters 88 and the breathing parameters 88 may be detected by sensors 210 in other ways, as would be readily recognized by those of ordinary skill in the art upon review of this disclosure.

The respiratory therapy apparatus 10 may initially deliver a comfortable sub-therapeutic pressure p_{B} to the user. The respiratory therapy apparatus 10 may increase the pressure 106 to the therapeutic pressure p_{T} in one or more pressure steps 100 delivered on demand. Demand may be indicated by the user's breath intervals 92. In various aspects, the pressure 106 may be increased from the sub-therapeutic pressure p_{B} to the therapeutic pressure p_{T} in a series of pressure steps 100 over a series of breath intervals 92 with each pressure step 100 occurring within a breath interval 92. Accordingly, the breathing parameters 88 may be generally indicative of or may be used to determine the user's breath interval 92 to allow the respiratory therapy apparatus 10 to deliver the pressure steps 100 within the breath intervals 92. The respiratory therapy apparatus may detect the breathing parameters 88 of the particular breaths 90 and deliver the pressure step 100 generally proximate the particular breathing parameters 88. For example, the respiratory therapy apparatus 10 may detect the inhalation initiation 91 and deliver the pressure step 100 generally proximate the inhalation initiation 91 of the breath 90.

The pressure step 100 represents an increase in the pressure 106 from a first pressure 104 to a second pressure 105. The pressure step 100 occurs over a time interval 102 generally less than the breath interval 92. The pressure 106 is only generally increased during the time interval 102 of the pressure step 100, and otherwise remains generally constant in some aspects, or may be allowed to decrease in other aspects over the remaining portions of the breath interval 92. In some aspects, one pressure step 100 is delivered per breath interval 92. In other aspects, a plurality of pressure steps 100 may be delivered per breath interval 92.

The time interval 102 is less than the breath interval 92 and may be substantially less than the breath interval 92. For example, the breath interval 92 may range from about 3 seconds to about 12 seconds with a typical value of about 5 seconds, and the time interval 102 may be on the order of tenths or hundredths of a second. The time interval 102 is within the breath interval 92 so that the pressure step 100 is initiated and terminated within the breath interval 92.

The time interval 102 may be determined from one or more breathing parameters 88. For example, the time interval 102 could be determined from the breath interval 92 of one or more previous breaths 90 in order to be less than the breath interval 92. In various aspects, the time interval 102 may be proportional to the previous breath interval 92 or may be proportional to a moving average of previous breath intervals 92. In other aspects the time interval 102 may be generally fixed about some constant value that is likely to be substantially less than a breath interval 92, for example, a few hundredths of a second.

In some aspects, the pressure step 100 delivered during the breath interval 92 of a particular breath 90 may be functionally related to the breathing parameters 88 of one or more previous breaths 90. For example, the pressure step 100 delivered during a particular breath 90 could be proportional to the breath interval 92 of the previous breath 90, so that a longer breath interval 92 for the previous breath 90 would result in a relatively larger pressure step 100, and a shorter breath interval 92 for the previous breath 90 would result in a relatively smaller pressure step 100. Accordingly, in this example, demand is indicated by the breath interval 92 of the previous breath 90, and demand is proportional to the breath interval 92 of the previous breath 90. Other functional relationships could be used in various aspects such as polynomial, logarithmic, power functions, and logic functions, and the demand could be functionally related to one or more breathing parameters 88. The breathing parameters 88 could encompass one or more previous breaths 90. In various aspects, the pressure step 100 could be delivered every other breath 90, every third breath 90, and so forth, and combinations thereof, or the pressure step 100 could be delivered at random or irregular breaths 90.

In some aspects, the pressure step 100 delivered during the breath interval 92 of a particular breath 90 may be functionally related to the breathing parameters 88 of the particular breath 90. For example, the respiratory therapy apparatus 10 may detect the inhalation peak 96 of the particular breath 90 and deliver the pressure step 100 generally proximate the inhalation peak 96 of that particular breath 90. The pressure step 100 may, for example, be related to the magnitude of the inhalation peak 96 of that particular breath 90.

In some aspects, the pressure step 100 may be chosen such that the pressure 106 increases from the sub-therapeutic pressure p_{B} to the therapeutic pressure p_{T} generally over a selected rise time 150. For example, the pressure step 100 may be chosen so that one pressure step 100 delivered per breath interval 92 results in an increase in the pressure 106 from the sub-therapeutic pressure p_{B} to the therapeutic pressure p_{T} generally in the rise time 150. The pressure step 100 may be constant or may vary in order to achieve the therapeutic pressure p_{T} over the selected rise time 150. In some aspects, the rise time 150 extends over several breath intervals 92 and may, in various aspects, be on the order of several minutes or even on the order of a half-hour.

In various aspects, the respiratory therapy apparatus 10 may be configured to detect the user's progress toward the sleep state 128 from the user's breathing parameters 88. The respiratory therapy apparatus 10 may be configured to control the pressure 106 to deliver a comfortable sub-therapeutic pressure p_{B} to the user initially, and then increase the pressure 106 from the sub-therapeutic pressure p_{B} to the therapeutic pressure p_{T} in one or more pressure steps 100 delivered on demand as the user progresses to the sleep state 128.

The demand may be determined from the user's progression toward the sleep state 128 and/or attainment of the sleep state 128. The user's breathing parameters 88 may be indicative of the user's progression toward the sleep state 128 and, accordingly, the demand may be determined from the breathing parameters 88. For example, while the user remains in a state of wakefulness 124, the pressure 106 may be maintained generally proximate the sub-therapeutic pressure p_{B}. The pressure steps 100 may be relatively small or essentially or actually no increase while the user is in a state of wakefulness 124. The pressure 106 may be increased to the therapeutic pressure p_{T}, usually in one or more pressure steps 100 as the user progresses toward the sleep state 128 and/or achieves the sleep state 128 as indicated by the breathing parameters 88. As the user progresses toward the sleep state 128, the pressure steps 100 are increased to deliver an increased pressure 106 to the user. Thus, a pressure 106 generally proximate the sub-therapeutic pressure p_{B} is delivered to the user during an initial period of wakefulness 124, while the therapeutic pressure p_{T} is delivered to the user generally proximate to the time that the user attains the sleep state 128. In various aspects, the respiratory therapy apparatus 10 increases the pressure 106 delivered to the user from the sub-therapeutic pressure p_{B} to the therapeutic pressure p_{T} based upon demand determined from the user's progress to and/or attainment of the sleep state 128 as indicated by the breathing parameters 88.

In various aspects, the demand for increased pressure as the user progresses to the sleep state 128 may be determined from the user's breath interval 92. For example, when the user first retires, the user's breath interval 92 may be relatively long meaning that the user's breathing rate is relatively slow. As the user relaxes and progresses toward the sleep state 128, the user's breath interval 92 may change, for example, by decreasing breath airflow amplitude 94 with corresponding decrease in the user's breath interval 92. The user may establish a regular short breath interval 92 upon falling asleep. Thus, the progression of the breath interval 92 from relatively long to relatively short may indicate the user's progress toward the sleep state 128.

The respiratory therapy apparatus 10 may increase the pressure by a pressure step 100 during a single breath 90 based upon the breath interval 92 or breath intervals 92 of one or more previous breaths 90 in various aspects. For example, if the previous breath interval 92 is relatively long, indicative of relaxed wakefulness 124, the pressure step 100 may be small or essentially zero to either maintain the pressure 106 or may otherwise be chosen to produce a relatively slow progression in the pressure 106 to the therapeutic pressure p_{T}. If the previous breath interval 92 is short, indicative of progression toward the sleep state 128, the pressure step 100 may be altered to increase the pressure 106 to the therapeutic pressure p_{T} and/or to increase the rate of progression to the therapeutic pressure p_{T}. In some aspects, the pressure step 100 may be chosen to achieve the therapeutic pressure p_{T} more or less immediately. For example, if the breath interval 92 exceeds the critical breath interval 136, this may be indicative of an apneic event, and the pressure step 100 may be chosen to achieve immediately the therapeutic pressure p_{T}.

Thus, the breathing parameters 88 may be indicative of normal breathing, and/or the breathing parameters 88 may be indicative of abnormal breathing such as an apnea or other abnormal breathing requiring therapy as would be understood by one of ordinary skill in the art upon review of this disclosure. In various aspects, the respiratory therapy apparatus 10 is configured to provide pressure steps 100 to increase the pressure 106 delivered to the user from the sub-therapeutic pressure p_{B} to the therapeutic pressure p_{T} based upon demand when the breathing parameters 88 are indicative of normal breathing. The respiratory therapy apparatus 10 may be configured to achieve the therapeutic pressure p_{T} immediately if abnormal breathing is detected.

The pressure step 100 may range from substantially zero to a maximum. For example, during periods of wakefulness 124, the pressure step 100 may be substantially zero. At the other limit, for example if an apneic event is detected, the pressure step 100 may be the maximum, which may be substantially equal to the difference between the therapeutic pressure p_{T} and the pressure 106 currently delivered to the user so that the pressure 106 steps up to the therapeutic pressure p_{T} more or less immediately.

In various aspects, the demand for increased pressure 106 delivered to the user as the user progresses to the sleep state 128 may be determined from the user's mean breath airflow amplitude 132. A decrease in the mean breath airflow amplitude 132 may be indicative of the user's progression toward the sleep state 128. The pressure step 100 may be determined from the mean breath airflow amplitude 132 of one or more previous breaths 90. The pressure step 100 would be small or essentially zero when the mean breath airflow amplitude 132 of the one or more previous breaths 90 is indicative of wakefulness 124. The pressure step 100 would be increased when the mean breath airflow amplitude 132 of one or more previous breaths 90 is indicative of progression toward the sleep state 128. Again, the breath amplitude 94 of the one or more previous breath intervals 90 may be indicative of an apneic event, and the pressure step 100 increased accordingly so that the pressure 106 more or less immediately achieves the therapeutic pressure p_{T}.

In still other aspects, the tidal volume 93 the breath volume 118, and/or the minute ventilation may be used to determine the user's progression or lack of progression toward the sleep state 128. As the user progresses toward the sleep state 128, the breath volume 118 decreases and the tidal volume 93 and the minute ventilation also decrease. Thus, the pressure step 100 may be increased when the breath volume 118 or the tidal volume 93 of the one or more previous breath intervals 90 is indicative of progression toward the sleep state 128. The pressure step 100 may be increase, in some aspects, when the minute ventilation decreases, which indicates progression toward the sleep state 128.

It should be recognized that various users may exhibit various patterns in breathing parameters 88 including breath interval 92, breath airflow amplitude 94, tidal volume 93, and breath volume 118 as they progress toward the sleep state 128, and that even a single user may exhibit variations in patterns of breathing parameters 88 as they progress toward the sleep state 128. Accordingly, the respiratory therapy apparatus 10 may detect these various patterns of breathing parameters 88 and may be adjustable to deliver pressure steps 100 based upon these various patterns. Thus, the respiratory therapy apparatus 10 may tune detection of wakefulness 124 and sleep state 128 to the particular user. In some aspects, tuning may be accomplished over a series of sleep episodes. A particular user may exhibit an anomalous pattern of breathing parameters 88 when progressing from wakefulness 124 to the sleep state 128 such as, for example, an increase in breath interval 92. In some aspects, the user and/or the health care professional may adjust the respiratory therapy apparatus 10 to detect the progression from wakefulness 124 to the sleep state 128 using such anomalous patterns of breathing parameters 88. In some aspects, the user and/or the health care professional may adjust the respiratory therapy apparatus 10 to be more responsive or less responsive to particular patterns of breathing parameters 88. In some aspects, artificial intelligence may be employed to tune the respiratory therapy apparatus 10.

In various other aspects, the demand for increased pressure 106 as the user progresses to the sleep state 128 may be determined from combinations of the user's breath interval 92, breath airflow amplitude 94, and tidal volume 93 as would be recognized by those of ordinary skill in the art upon review of this disclosure. Breathing parameters 88 measured over a plurality of prior breath intervals 90 may be used to determine the demand. Rates of change of the breathing parameters 88, integral measures of the breathing parameters 88, and various statistical measures of breathing parameters 88 such as moving averages may also be employed alone or in combination in various aspects to determine the demand, as would be recognized by those of ordinary skill in the art upon review of this disclosure. Other breathing parameters 88, combinations of breathing parameters 88, and measures derived from the breathing parameters 88 indicative of the progression from wakefulness 124 to the sleep state 128 may also be used to determine the demand for increased pressure as would be recognized by those of ordinary skill in the art upon review of this disclosure. The pressure step 100 could be increased in correspondence to the combinations of breath interval 92 and breath airflow amplitude 94 and/or in correspondence with the other breathing parameters 88 indicative of progression toward the sleep state 128. Indications of an apneic event could cause the pressure step 100 to be set to more or less immediately achieve the therapeutic pressure p_{T}. In some aspects, the pressure 106 may be decreased in one or more pressure steps 100 each delivered within one breath interval 92 if return to wakefulness 124 is detected.

The time interval 102 of the pressure step 100 is the time required for the pressure step 100 to occur, i.e. the time required for the pressure 106 to increase by the amount of the pressure step 100. The time interval 102 of the pressure step 100 may be generally less than or equal to the breath interval 92 of a single breath 90 so that the pressure step 100 occurs generally within a breath interval 92. The pressure step 100 may coincide with various portions of the breath 90 depending upon the pressure step 100 and the time interval 102 of the pressure step 100. For example, in various aspects, the pressure step 100 may be provided as a rapid step increase in pressure 106 generally during the inhalation 95 portion of the breath 90 to provide additional airflow at increased pressure during inhalation 95. The time interval 102 over which a rapid step increase occurs may be generally limited by the time required for the respiratory therapy apparatus 10 to increase the pressure 106, which may be related, for example, to the inertia of various mechanical components of the flow generator 20 such as an electric motor and/or a fan blade. In some aspects, the pressure step 100 may be provided as a rapid step increase in pressure 106 generally proximate the inhalation peak 96. The pressure step 100 could, in some aspects be provided as a rapid step increase in pressure 106 at the inhalation initiation 91 of the breath 90. In some aspects, the pressure step 100 could be a pressure increase generally over at least portions of the inhalation 95 portion of the breath 90 so that the time interval 102 is at least somewhat greater than the time interval 102 of a rapid step increase. Such a pressure step 100 could have, in some aspects, the form of a generally linear increase in pressure 106 with respect to time, and could have the form of a parabolic increase in pressure 106 with respect to time in other aspects. The pressure step 100 could show other forms of increase over the time interval 102 in various other aspects. The pressure step 100 may be provided over other portions of the breath 90 and may have various forms, and the pressure step 100 may be otherwise configured as would be recognized by those of ordinary skill in the art upon review of this disclosure.

Following the pressure step 100, the pressure 106 may be maintained generally constant over the remainder, if any, of the breath interval 92 in some aspects. In other aspects, the pressure may be allowed to decrease by pressure drop 144 over at least portions of the remainder of the breath interval 92. For example, the pressure step 100 may be delivered as a rapid step increase in pressure at the beginning of the inhalation 95 portion of the breath 90 with the pressure maintained constant over the remainder of the inhalation 95 portion of the breath 90. The pressure may then be decreased generally during the exhalation 99 portion of the breath 90 by pressure drop 144.

The pressure steps 100 may be provided during each breath 90 in succession in some aspects. In other aspects, the pressure steps 100 may be provided every other breath 90, every third breath 90, and so forth, or randomized. Combinations thereof may be provided in some aspects. For example, when wakefulness 124 is detected, the pressure step 100 may be provided every third breath 90, and, as the user progresses toward the sleep state 128, the pressure step 100 may be provided every second breath 90 and, thence, every breath 90 until the therapeutic pressure p_{T} is attained. The timing of the pressure step 100 may be sequenced to the timing of one or more previous breath intervals 92 so that the pressure step 100 is generally provided during an appropriate portion of the breath interval 92. In some aspects, the pressure 106 may be maintained at the sub-therapeutic pressure p_{B} for a number of breaths 90 and then increased to the therapeutic pressure p_{T} in one or more pressure steps 100.

In operation, the pressure 106 of the pressurized air delivered to the user by the respiratory therapy apparatus 10 may be increased from the sub-therapeutic pressure p_{B} to the therapeutic pressure p_{T} in one or more pressure steps 100. The pressure steps 100 may be based upon user demand. In some aspects, the user demand may be based upon the user's breathing parameters. In other aspects, the user demand may be based upon the user's progress toward the sleep state 128 as determined from the breathing parameters 88. One or more sensors 210 may be provided in the respiratory therapy apparatus 10 to detect the user's breathing parameters 88 and to generate signals 212 indicative of the user's breathing parameters 88. In various aspects, the control unit 26 receives the signals 212 indicative of the user's breathing parameters 88 from the one or more sensors 210. The control unit 26 then utilizes these signals 212 to formulate one or more control signals 214 which are then communicated to one or more components within the respiratory therapy apparatus 10 to control the pressure 106 delivered to the user. The timing of the pressure step 100 in relation to the breath 90 and the form of the pressure step 100 may be determined by the control unit 26.

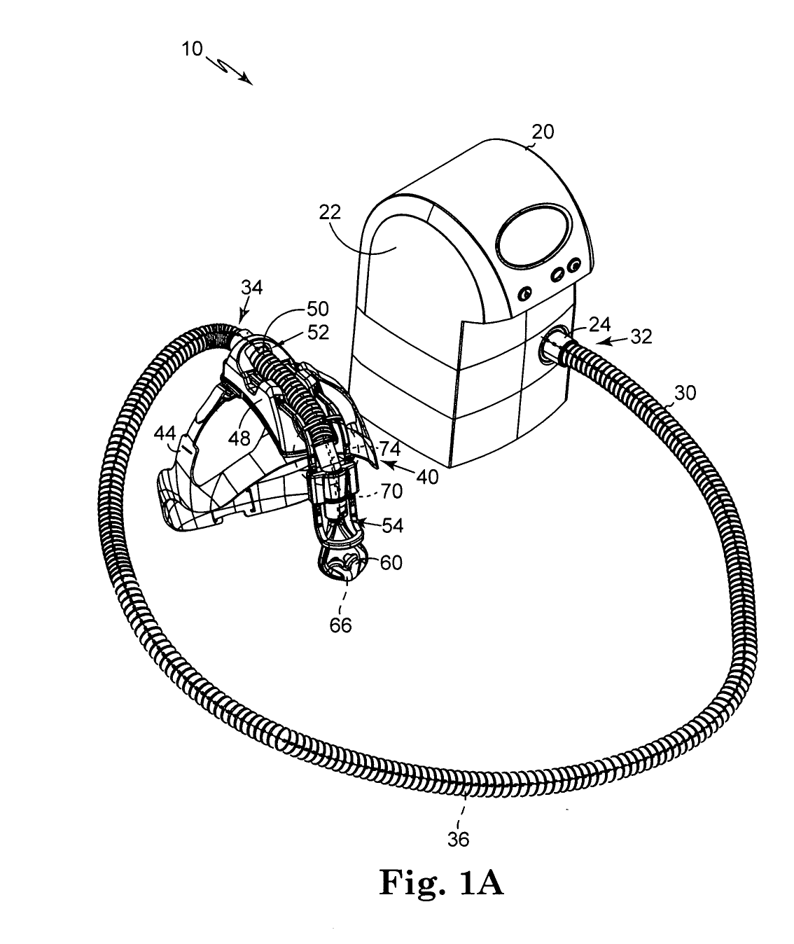

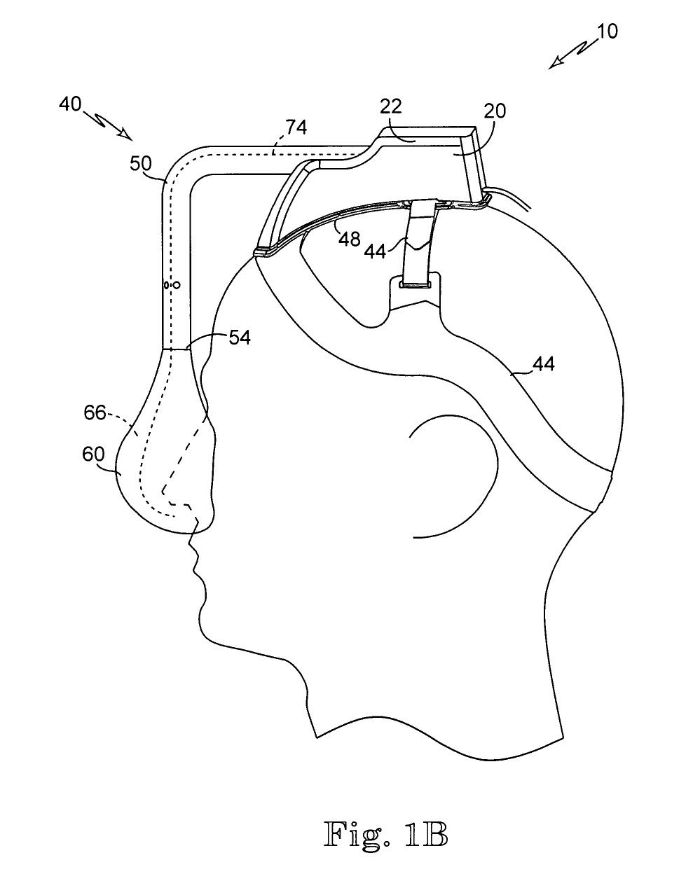

Specific exemplary embodiments of the respiratory therapy apparatus 10 are illustrated in the Figures. FIG. 1A generally illustrates an embodiment of the respiratory therapy apparatus 10. As illustrated, the respiratory therapy apparatus 10 includes a flow generator 20, a user interface 40, and a delivery tube 30. The flow generator 20 includes an outlet 24 through which pressurized air generated by the flow generator 20 may pass. The user interface 40 includes an interface conduit 50 and a mask 60. The user interface, as illustrated, also includes various support structures including a mount 48 and support bands 44 to secure the user interface 40 about the user's head and properly position the mask 60 with respect to the user.

The interface conduit has an interface conduit proximal end 52, and interface conduit distal end 54, and defines interface passage 74. The interface conduit distal end 54 is secured to mask 60 such that the interface passage 74 is in fluid communication with a chamber 66 defined by the mask 60. The delivery tube 30 defines a delivery tube passage 36, and the proximal end 32 of the delivery tube 30 may be attached to the outlet 24 of the flow generator 20, as illustrated in FIG. 1A. The distal end 34 of the delivery tube 30 may be secured to the interface conduit proximal end 52 such that pressurized air may be delivered from the outlet 24 of the flow generator 20 through the delivery tube passage 36 and through the interface passage 74 and into the chamber 66 of the mask 60 for inhalation by the user. In the embodiment illustrated in FIG. 1A, the mask 60 is configured to be sealed about the user's nares and to touch the user's face generally proximate the nares.

Another embodiment of the respiratory therapy apparatus 10 is illustrated in FIG. 1B. The embodiment illustrated in FIG. 1B includes a flow generator 20 that is attached to the user interface 40 generally about the mount 48. The mount 48 provides a generally rigid structure to which portions of the user interface 40 including the flow generator 20, portions of the interface conduit 50, and one or more of the support bands 44 may be secured. A plurality of support bands 44 are provided to secure the user interface 40 including the flow generator 20 about the user's head. Pressurized air may be communicated from the flow generator 20 through interface passage 74 defined by interface conduit 50 to the chamber 66 of mask 60. The mask 60, in this embodiment, may be sealed about the user's nares to deliver pressurized air for inhalation by the user. The interface conduit 50 is shown as extending from the flow generator 20 housing 22 and bending to pass over the user's face without touching the user's face and is generally in a fixed orientation with respect to the user's head including the face.

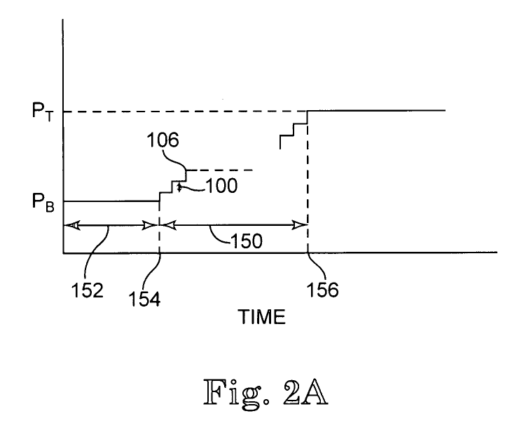

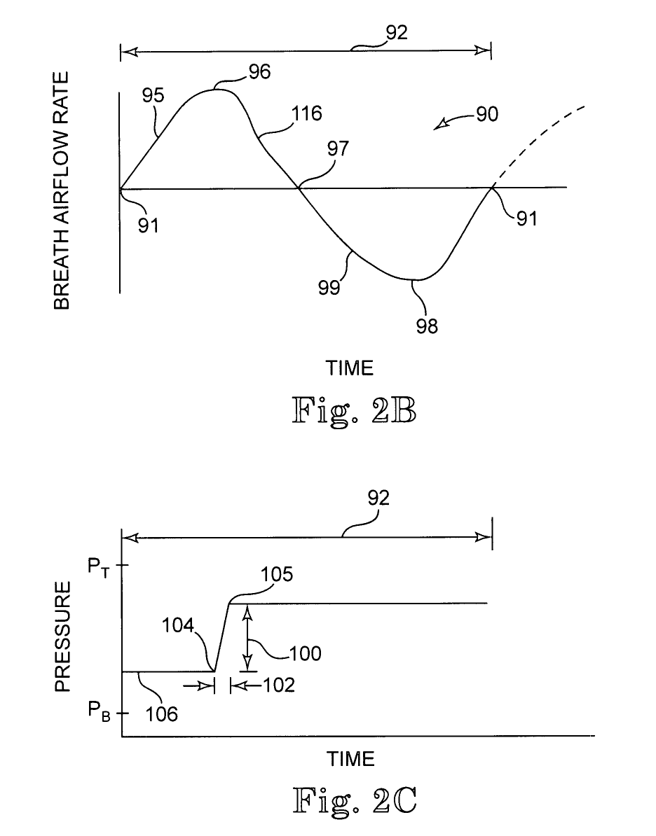

The respiratory therapy apparatus may increase the pressure 106 from the sub-therapeutic pressure p_{B} to the therapeutic pressure p_{T} in one or more pressure steps 100 as illustrated in FIG. 2. As illustrated in FIG. 2, the pressure 106 may be maintained generally constant about the sub-therapeutic pressure p_{B} for an initiation time 152 that may commence at the powering up of the respiratory therapy apparatus 10. During the initiation time 152, the user may secure the mask about his/her head and face and retire to bed.

At the rise initiation time 154, which marks the end of the initiation time 152 and the beginning of the rise time 150, the pressure 106 begins its increase from the sub-therapeutic pressure p_{B} to the therapeutic pressure p_{T} in one or more pressure steps 100. The user may then, in some embodiments, select the rise initiation time 154 by, for example, pushing a button on the flow generator housing 22 to signal the control unit 26 to initiate the increase in the pressure 106 from the sub-therapeutic pressure p_{B} to the therapeutic pressure p_{T}. In other embodiments, the rise initiation time 154 may be pre-selected to be initiated by the control unit 26.

Beginning at the rise initiation time 154, the respiratory therapy apparatus 10 may then increase the pressure 106 from the sub-therapeutic pressure p_{B} to the therapeutic pressure p_{T} in one or more pressure steps 100, each pressure step 100 occurring over time interval 102. The time interval 102 is less than the breath interval 92, and, in some embodiments, the time interval 102 is substantially less than the breath interval 92. In some embodiments, the pressure 106 may be increased by a single pressure step 100 during a particular breath interval 92. In other embodiments, the pressure 106 may be increased by two pressure steps 100 during a particular breath interval 92. In still other embodiments, the pressure 106 may be increased by three or more pressure steps 100 during a particular breath interval 92. The respiratory therapy apparatus 10, as illustrated in FIG. 2, delivers the therapeutic pressure p_{T} to the user at the therapy time 156. The rise time 150 is the time required to increase the pressure 106 from the sub-therapeutic pressure p_{B} to the therapeutic pressure p_{T}, as illustrated. In some embodiments, the rise time 150 may be pre-selected and the pressure steps 100 chosen to increase the pressure 106 from the sub-therapeutic pressure p_{B} to the therapeutic pressure p_{T} over the pre-selected rise time 150. In other embodiments, the rise time 150 may be a function of demand.

In the embodiment illustrated in FIGS. 2B and 2C, the respiratory therapy apparatus 10 is configured to deliver a single pressure step 100 within the breath interval 92. As illustrated in FIG. 2B, the breath 90 begins with inhalation initiation 91 at the start of an inhalation 95 and includes inhalation 95. The breath 90 continues to exhalation initiation 97 and includes exhalation 99 to the completion of exhalation 99 and the inhalation initiation 91 of the succeeding breath 90. The breath interval 92, in this embodiment, is the time required for one breath 90 including the inhalation 95 followed by exhalation 99.

As illustrated in FIG. 2C, the pressure step 100 represents an increase in the pressure 106 from the first pressure 104 to the second pressure 105. The pressure step 100 occurs over the time interval 102, which is generally less than the breath interval 92. The pressure 106 is only generally increased during the time interval 102 of the pressure step 100, and otherwise remains generally constant in this embodiment at either the first pressure 104 or the second pressure 105 over the portions of the breath interval 92 outside of the time interval 102.

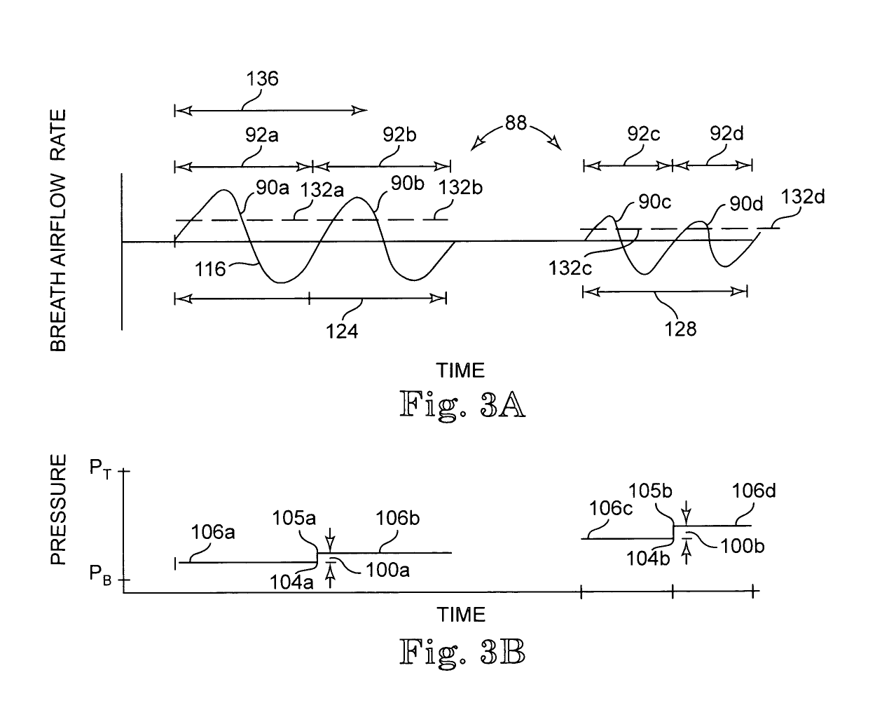

The respiratory therapy apparatus may increase the pressure 106 from the sub-therapeutic pressure p_{B} to the therapeutic pressure p_{T} in one or more pressure steps 100 delivered on demand where demand is indicated by the breath interval 92 of the previous breath 90, as illustrated in FIGS. 3A and 3B. As illustrated, the breath intervals 92a, 92b of breaths 90a, 90b are relatively long in comparison with the breath intervals 92c, 92d of breaths 90c, 90d. In this embodiment, the breath intervals 92a, 92b, 92c, 92d are less than the critical breath interval 136, which may generally indicate that the user is breathing normally. The pressure step 106, in this embodiment, is proportional to the breath interval 92 of the previous breath 90. Accordingly, the pressure step 100a is proportional to the breath interval 92a and the pressure step 100b is proportional to the breath interval 92c so that the pressure step 100a is relatively small in comparison to the pressure step 100b in this illustration.

If the breath intervals 92a, 92b, 92c, 92d become greater than the critical breath interval 136, this may be indicative of abnormal breathing including apnea. Accordingly, the proportional relationship between the pressure step 100 and the breath interval 92 may be abandoned and one or more pressure steps 100 provided to increase the pressure 106 generally immediately to the therapeutic pressure p_{T}.

In various embodiments, the pressure step 100 may be functionally related to the previous breath interval 92, and the functional relationship may vary depending upon the length of the previous breath interval 92. For example, the pressure step 100 could be functionally related to the breath interval 92 according to the functional relationship given by the power function:

Δpᵢ=k(Iᵢ₋₁)^{m}/60 (1)

where:

Δpᵢ is the pressure step 100 delivered during the i^{th} breath interval 92

Iᵢ₋₁ is the breath interval 92 of the (i−1)^{th} breath 90,

m is the exponent of the power function,

k is a sensitivity factor.

The time required to achieve an increase of 10 cm of H₂O in the pressure 106 for various constant breath intervals 92 is tabulated in Table 1 for equation (1) using various values of k and m. In this example, the values of k were chosen so that a breath interval 92 of 6 seconds (equivalent to 10 breaths per minute) would produce a 10 cm of H₂O pressure increase in 30 minutes.

As indicated in Table 1, the time required to achieve an increase of 10 cm of H₂O in the pressure 106 for m=¼ varies from 50.4 minutes for a breath interval 92 of 12 seconds to 17.8 minutes for a breath interval 92 of 3 seconds. Longer breath intervals 92 may be indicative of wakefulness 124 and shorter breath intervals 92 may be indicative of the sleep state 128. The time required to achieve an increase of 10 cm of H₂O in the pressure 106 for m=½ varies from 21.2 minutes for a breath interval 92 of 3 seconds to 42.5 minutes for a breath interval 92 of 12 seconds. The time required to achieve an increase of 10 cm of H₂O in the pressure 106 for m=−1 varies from 7.5 minutes for a breath interval 92 of 3 seconds to 120 minutes for a breath interval 92 of 12 seconds in this example. Thus, in this example, the values of m and k are chosen so that the pressure 106 increases relatively slowly during wakefulness 124 and increases relatively rapidly as the user approaches and/or attains the sleep state 128.

Note that some choices of m and k may result in a larger pressure step 100 for larger breath intervals 92 but also require more time to achieve an increase of 10 cm of H₂O in the pressure 106 when the breath intervals 92 are larger because the larger pressure step is delivered over fewer breath intervals 92 per unit time.

TABLE 1

| | | Δpᵢ = 0.20 | Δpᵢ = 0.816 | Δpᵢ = 1.28 |
|---|---|---|---|---|
| | | (Iᵢ₋₁)⁻¹/60 | (Iᵢ₋₁)^{1/2}/60 | (Iᵢ₋₁)^{1/4}/60 |
| | | Time (min) for 10 cm | Time (min) for 10 cm | Time (min) for 10 cm |
| Breaths per minute | | H₂O pressure | H₂O pressure | H₂O pressure |
| (BPM) | Breath Interval (s) | change | change | change |
| 20 | 3 | 7.5 | 21.2 | 17.8 |
| 15 | 4 | 13.3 | 24.5 | 22.1 |
| 12 | 5 | 20.8 | 27.4 | 26.1 |
| 10 | 6 | 30.0 | 30.0 | 30.0 |
| 7.5 | 8 | 53.3 | 34.7 | 37.2 |
| 6 | 10 | 83.3 | 38.8 | 43.9 |
| 5 | 12 | 120.0 | 42.5 | 50.4 |

In various embodiments, it may be beneficial to alter the functional relationship between pressure step 100 and the breath interval 92 so that, for example, the pressure 100 increases more rapidly with increasing breath interval 92 when the breath interval 92 exceeds the critical breath interval 136. For example, the critical breath interval 136 may be about 6 seconds, as breath intervals 92 greater than about 6 seconds may be indicative of abnormal breathing including apnea. Thus, in some exemplary embodiments, when breath intervals 92 greater than the critical breath interval 136 are detected, the respiratory therapy apparatus 10 may increase the pressure 106 to the therapeutic pressure p_{T} using pressure steps 100 given by equation (1) with k and m chosen so that the pressure 106 increases more rapidly with increasing breath interval 92. Table 2 illustrates the relationship between breath interval 92 and the time required to increase the pressure 106 by 10 cm of H₂O for values of k and m using equation (1) so that the pressure 106 increases more rapidly for increasing breath intervals 92.

The time required to achieve an increase of 10 cm of H₂O in the pressure 106 for various constant breathing rates is tabulated in Table 2 for equation (1) using various values of k and m. In this example, the values of k were chosen so that a breathing rate of 10 breaths per minute would produce a 10 cm of H₂O pressure increase in 30 minutes.

As the exponent m in equation (1) is increased, the sensitivity of Δpᵢ to Iᵢ₋₁ increases, as illustrated by the example in Table 2. As indicated in Table 2, the time required to achieve an increase of 10 cm of H₂O in the pressure 106 for m= 3/2 varies from 42.5 minutes for a breath interval 92 of 3 seconds to 21.2 minutes for a breath interval 92 of 12 seconds. The time required to achieve an increase of 10 cm of H₂O in the pressure 106 for m=2 varies from 60.0 minutes for a breath interval 92 of 3 seconds to 15.0 minutes for a breath interval 92 of 12 seconds. The time required to achieve an increase of 10 cm of H₂O in the pressure 106 for m=3 varies from 120 minutes for a breath interval 92 of 3 seconds to 7.5 minutes for a breath interval 92 of 12 seconds in this example.

Thus, in some embodiments, the pressure step 100 may be functionally related to the breath interval 92 as indicated by equation (1). The constant k and the exponent m of the power function may be alterable between first values k₁ and m₁ and second values k₂ and m₂. The constant k and the exponent m are set to the first values k₁ and m₁ when the breath interval is generally less than a critical breath interval 136, the constant k and the exponent m are set to the second values k₂ and m₂ when the breath interval is generally greater than the critical breath interval 136. The values of k₁ and m₁ may be chosen so that the pressure 100 increases more rapidly as the breath interval 92 decreases, which may be indicative of the user's progress from wakefulness 124 to the sleep state 128 when the breath interval 92 is less than a critical breath interval 136. When the breath interval 92 exceeds the critical breath interval 136, this may be indicative of apnea and/or other breathing problems. The values of k₂ and m₂ used in equation (1) may be chosen so that the pressure 106 increases more rapidly as the breath interval 92 increases.

TABLE 2

| | | Δpᵢ = 0.136 | Δpᵢ = 0.055555 | Δpᵢ = 0.0092594 |
|---|---|---|---|---|
| | | (Iᵢ₋₁)^{3/2}/60 | (Iᵢ₋₁)²/60 | (Iᵢ₋₁)³/60 |
| | | Time (min) for 10 cm | Time (min) for 10 cm | Time (min) for 10 cm |
| Breaths per minute | | H₂O pressure | H₂O pressure | H₂O pressure |
| (BPM) | Breath Interval (s) | change | change | change |
| 20 | 3 | 42.5 | 60.0 | 120.0 |
| 15 | 4 | 36.8 | 45.0 | 67.5 |
| 12 | 5 | 32.9 | 36.0 | 43.2 |
| 10 | 6 | 30.0 | 30.0 | 30.0 |
| 7.5 | 8 | 26.0 | 22.5 | 16.9 |
| 6 | 10 | 23.3 | 18.0 | 10.8 |
| 5 | 12 | 21.2 | 15.0 | 7.5 |

In various embodiments, the pressure step 100 may be functionally related to the tidal volume 93 of the previous breath 90. For example, the pressure step 100 could be functionally related to the tidal volume 93 according to the functional relationship given by the power function:

Δpᵢ=c(TVᵢ₋₁)ⁿ/60 (2)

where:

Δpᵢ is the pressure step 100 delivered during the i^{th} breath interval 92

TVᵢ₋₁ is the tidal volume 93 of the (i−1)^{th} breath 90,

n is the exponent of the power function,

c is a sensitivity factor.

The sensitivity of the pressures step 100 to the tidal volume 93 may be adjusted by adjusting the exponent n in equation 2. As indicated in Table 3, the time required to achieve an increase of 10 cm of H₂O in the pressure 106 for n=−2 varies from 13.3 minutes for a tidal volume 93 of 300 ml, which may be indicative of the sleep state 128, to 53.3 minutes for a tidal volume 93 of 600 ml, which may be indicative of wakefulness 124. The time required to achieve an increase of 10 cm of H₂O in the pressure 106 for n =−½ varies from 24.5 minutes for a tidal volume 93 of 300 ml to 34.6 minutes for a tidal volume 93 of 600 ml. The time required to achieve an increase of 10 cm of H₂O in the pressure 106 for n=−1 varies from 20.0 minutes for a tidal volume 93 of 300 ml to 40.0 minutes for a tidal volume 93 of 600 ml in the example of Table 3. Thus, in this example, the values of n and c are chosen so that the pressure 106 increases relatively slowly during wakefulness 124 and increases relatively rapidly as the user approaches and/or attains the sleep state 128.

TABLE 3

| | | | | | | Δpᵢ = 270,000 |
|---|---|---|---|---|---|---|
| | | | | Δpᵢ = 600 | Δpᵢ = 28.3 | (TVᵢ₋₁)⁻²/60 |
| | | | | (TVᵢ₋₁)⁻¹/60 | (TVᵢ₋₁)^{-1/2}/60 | Time (min) for |
| Breaths per | | Tidal | Minute | Time (min) for | Time (min) for | 10 cm H₂O |
| minute | Breath | Volume | Ventilation | 10 cm H₂O | 10 cm H₂O | pressure |
| (BPM) | Interval (s) | (ml) | (LPM) | pressure change | pressure change | change |
| 15 | 4 | 600 | 9,000 | 40.0 | 34.6 | 53.3 |
| 15 | 4 | 550 | 8,250 | 36.7 | 33.1 | 44.8 |
| 15 | 4 | 500 | 7,500 | 33.3 | 31.6 | 37.0 |
| 15 | 4 | 450 | 6,750 | 30.0 | 30.0 | 30.0 |
| 15 | 4 | 400 | 6,000 | 26.7 | 28.3 | 23.7 |
| 15 | 4 | 350 | 5,250 | 23.3 | 26.4 | 18.1 |
| 15 | 4 | 300 | 4,500 | 20.0 | 24.5 | 13.3 |

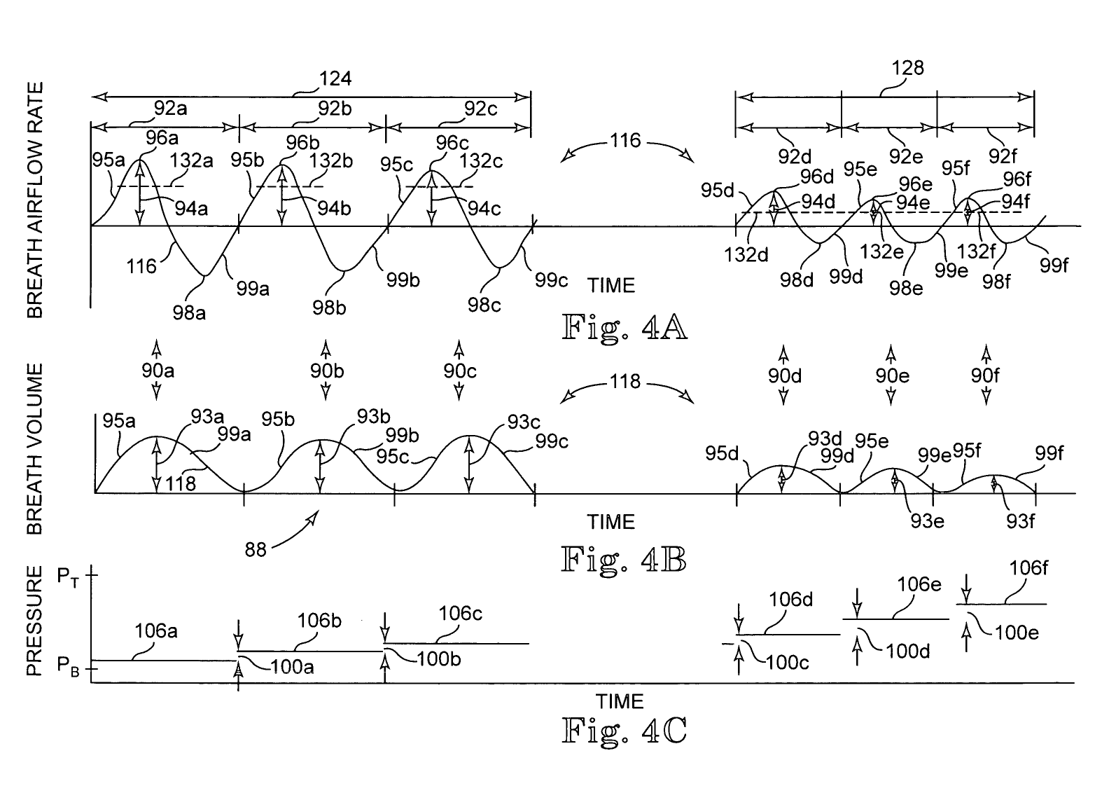

FIGS. 4A and 4B illustrates a time sequence of a plurality of breaths 90 designated 90a . . . 90f and corresponding breath intervals 92 designated 92a . . . 92f. The corresponding pressure steps 100a . . . 100e in the pressure 106a . . . 106f delivered to the user by the respiratory therapy apparatus 10 are illustrated in FIG. 4C. The breathing parameters 88 indicative of the breaths 90 are the breath airflow rate 116, illustrated in FIG. 4A, and the breath volume 118 illustrated in FIG. 4B. The breath airflow rate 116 is the flow rate of the pressurized air into and out of the user's air passages as the user inhales and exhales. The breath airflow amplitude 94 may be defined as the maximum breath airflow rate 116 during inhalation 95.

The breath volume 118 is the volume of pressurized air that passes into and out of the user's air passages as the user inhales and exhales. The breath volume 118 is the time integral of the breath airflow rate 116. The tidal volume 93 is the breath volume 118 passed into the user's air passages during inhalation 95. Exhalation empties the tidal volume 93 so that the breath volume 118 is essentially zero over a complete breath 90.

As illustrated in FIG. 4A, the breathing parameters 88 of the user indicate a period of wakefulness 124 followed by a period during when the user is approaching the sleep state 128 and/or has attained the sleep state 128. The period of wakefulness 124 may be indicated by relatively long breath intervals 92a, 92b, 92c in comparison with the relatively shorter breath intervals 92d, 92e, 92f during the period when the user is approaching the sleep state 128 and/or has attained the sleep state 128.

The breath airflow amplitude 94, in this illustration, is defined as the magnitude of the inhalation peak 96. As illustrated, the period of wakefulness 124 is indicated by relatively larger breath airflow amplitudes 94a, 94b, 94c in comparison to the relatively shallower breath airflow amplitudes 94d, 94e, 94f during the period when the user is approaching the sleep state 128 and/or has attained the sleep state 128. The period of wakefulness 124 may be indicated by relatively larger mean breath airflow amplitudes 132a, 132b, 132c in comparison with the relatively smaller mean breath airflow amplitudes 132d, 132e, 132f when the user is approaching the sleep state 128 and/or has attained the sleep state 128, as illustrated.

As illustrated in FIG. 4B, the period of wakefulness 124 and the period when the user is approaching the sleep state 128 or has attained the sleep state 128 may be indicated by the relatively larger tidal volumes 93a, 93b, 93c during wakefulness 124 in comparison with the tidal volumes 93d, 93e, 93f when the user is approaching of has attained the sleep state 128. The minute ventilation may also be used in various embodiments, with relatively larger minute ventilation indicative of wakefulness 124 and a relative decrease in minute ventilation indicative of the approach to or attainment of the sleep state 128.

As illustrated in FIG. 4C, the respiratory therapy apparatus 10 detects the period of wakefulness 124 and the period when the user is approaching the sleep state 128 and/or has attained the sleep state 128 from the breathing parameters 88. The respiratory therapy apparatus 10 increases the pressure 106a, 106b, 106c delivered to the user during the period of wakefulness 124 by relatively small pressure steps 100a, 100b in comparison with the pressure steps 100c, 100d, 100e in the pressure 106d, 106e, 106f delivered to the user during the period when the user is approaching the sleep state 128 and/or has attained the sleep state 128. Accordingly, the pressure 106 delivered to the user remains generally proximate the sub-therapeutic pressure p_{B} during the period of wakefulness 124, and the pressure 106 delivered to the user increases to the therapeutic pressure p_{T} during the period when the user is approaching the sleep state 128 and/or has attained the sleep state 128, as illustrated in FIG. 4C.

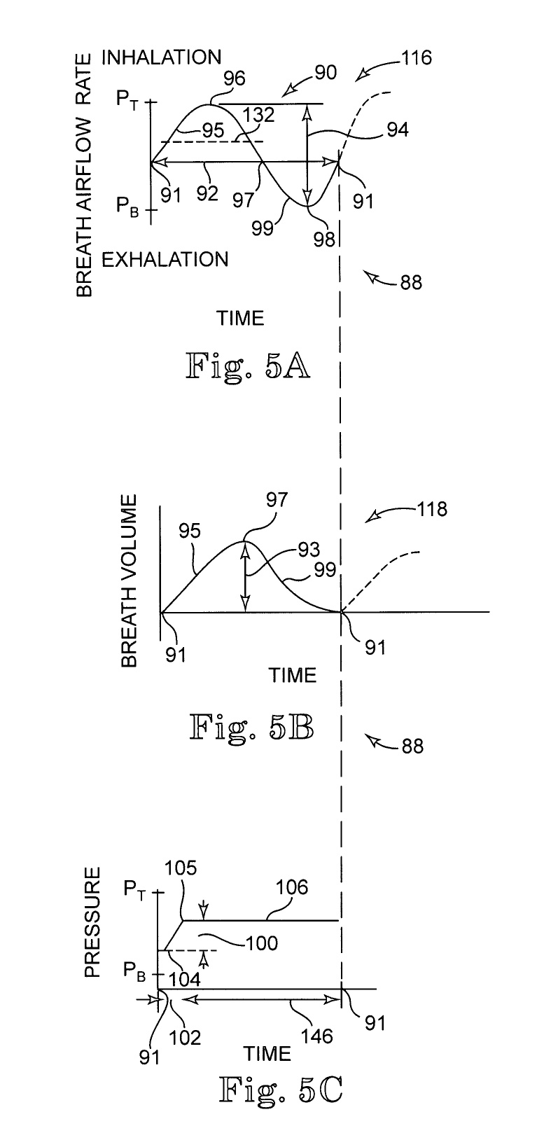

The breath 90 and the corresponding pressure step 100 for a specific embodiment of the respiratory therapy apparatus 10 are illustrated in FIGS. 5A, 5B, and 5C. As illustrated in FIG. 5A, the breath 90 includes inhalation 95 followed by exhalation 99. The breath 90 begins with the inhalation initiation 91, proceeds to the inhalation peak 96, to exhalation initiation 97, to the exhalation peak 98, and to the inhalation initiation 91 of the next breath 90. As illustrated in FIG. 5B, the breath 90 begins with inhalation 95. The breath volume 118 reaches a maximum, the tidal volume 93, as inhalation 95 is completed at exhalation initiation 97. The breath volume 118 then decreases until exhalation 99 is complete, as illustrated.

The breathing parameters 88, as illustrated, include the breath airflow amplitude 94 of the breath 90 measured as the difference between the inhalation peak 96 and the exhalation peak 98. The breathing parameters 88 also include the breath interval 92, the mean breath airflow amplitude 132, and the tidal volume 93. The breath interval 92 may be monitored using either the breath interval airflow rate 116 illustrated in FIG. 5A or from the breath volume 118 illustrated in FIG. 5B.

In the embodiment, of FIGS. 5A, 5B, and 5C, the pressure 106 is increased from the first pressure 104 to the second pressure 105 during breath interval 92 by pressure step 100. The pressure step 100 is delivered generally proximate the inhalation initiation 91 over time interval 102. The time interval 102 is small in comparison with the breath interval 92, so that the pressure step 100 generally has the form of a rapid step increase in pressure 106 in this embodiment. The pressure step 100 is provided generally coincident with inhalation 95, particularly between inhalation initiation 91 and prior to the inhalation peak 96 in this embodiment. The pressure 106 then remains generally constant at the second pressure 105 over the maintenance interval 146, which includes the portion of the breath 90 that follows the pressure step 100, as illustrated.

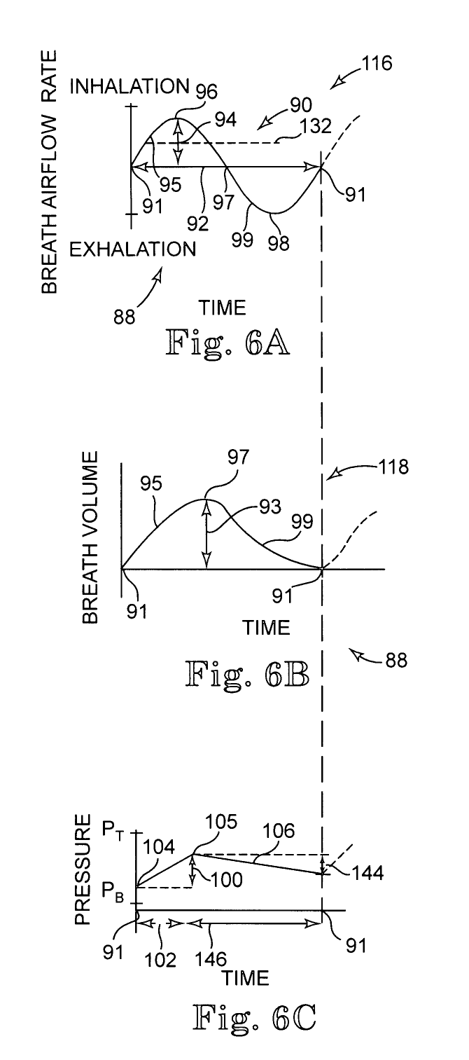

FIGS. 6A, 6B, and 6C illustrate breath 90 and the corresponding pressure step 100, respectively, for a specific embodiment of the respiratory therapy apparatus 10. As illustrated in FIG. 6A, the breath 90 includes inhalation 95 followed by exhalation 99. The breath 90 begins with the inhalation initiation 91, proceeds to the inhalation peak 96, to exhalation initiation 97, to the exhalation peak 98, and to the inhalation initiation 91 of the next breath 90, as illustrated in FIG. 6A. The breath volume 118 of breath 90 increases during inhalation 95 between inhalation initiation 91 and exhalation initiation 97 and then decreases until the inhalation initiation 91 of the next breath 90, as illustrated in FIG. 6B.

The breathing parameters 88, as illustrated, include the amplitude 94 of the breath 90 measured as the root mean square of the difference between the inhalation peak 96 and the exhalation peak 98 in this illustration. The breathing parameters 88 in this illustration include the breath interval 92, the mean breath airflow amplitude 132, and the tidal volume 93.

The pressure 106, in the embodiment of FIGS. 6A-6C, is increased from the first pressure 104 to the second pressure 105 by pressure step 100 generally proximate the inhalation initiation 91, and the pressure step 100 occurs over time interval 102 at least somewhat greater than the time interval 102 of the rapid step increase illustrated in FIGS. 5A-5C, so that the pressure step 100 has the form of a generally linear increase in pressure with respect to time in the embodiment of FIGS. 6A-6C. The pressure step 100 is provided generally coincident with inhalation 95, particularly between inhalation initiation 91 and the inhalation peak 96 in this embodiment. The pressure 106 then decreases from the second pressure 105 by pressure drop 144 over the maintenance interval 146, which includes the portion of the breath interval 92 following the pressure step 100, as illustrated in FIG. 6C. The pressure drop 144 is generally less than the pressure step 100 in this illustration.

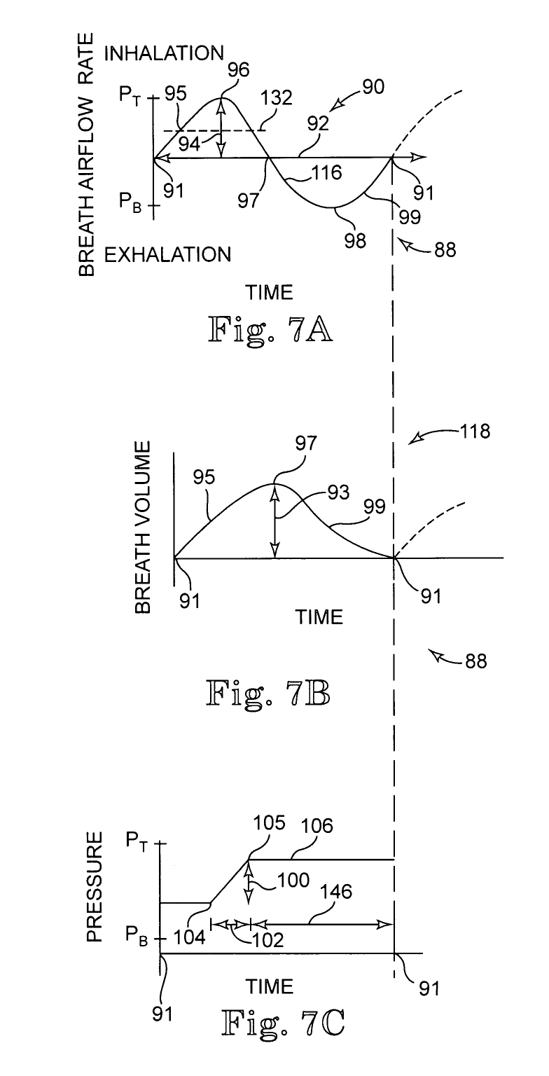

FIGS. 7A, 7B, and 7C illustrate breath 90 and the corresponding pressure step 100, respectively, for yet another embodiment of the respiratory therapy apparatus 10. As illustrated in FIG. 7A, the breath 90 includes inhalation 95 followed by exhalation 99. The breath 90 begins with the inhalation initiation 91, proceeds to the inhalation peak 96, to exhalation initiation 97, to the exhalation peak 98, and to the inhalation initiation 91 of the next breath 90, as illustrated in FIG. 7A. The breath volume 118 of breath 90 increases during inhalation 95 between inhalation initiation 91 and exhalation initiation 97 and then decreases until the inhalation initiation 91 of the next breath 90, as illustrated in FIG. 7B. The breathing parameters 88, as illustrated, include the amplitude 94 of the breath 90 measured as the amplitude of the inhalation peak 96. The breathing parameters 88 also include the breath interval 92, the tidal volume 93, and the mean breath airflow amplitude 132.

The pressure 106 is increased by pressure step 100 generally proximate the inhalation peak 96. The pressure step 100 occurs over time interval 102 so that the pressure step 100 generally has the form of a rapid step increase in pressure 106 in the embodiment of FIGS. 7A-7C. The pressure 106 then remains generally constant at the second pressure 105 over the maintenance interval 146, as illustrated.

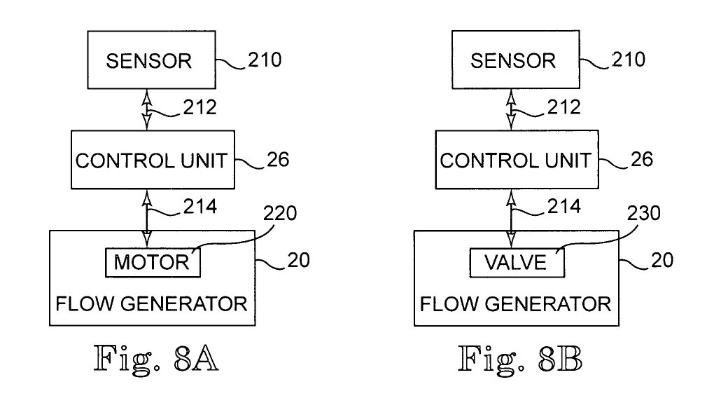

FIG. 8A illustrates an exemplary embodiment of the control unit 26 in the respiratory therapy apparatus 10. In this exemplary embodiment, the control unit 26 receives signals 212 indicative of the user's breathing parameters 88 from the sensor 210. The control unit 26 then utilizes these signals 212 to formulate a control signal 214 which is then communicated to the motor 220 within the flow generator 20 to control the pressure 106 delivered to the user. The control signal 214, for example, may direct the motor 220 to increase rotational speed in order to increase the pressure 106 by pressure step 100. The form of the pressure step 100 and the timing of the pressure step 100 in relation to the breath 90 would be determined by the timing of the increase in rotational speed of the motor 220 as controlled by the control unit 26 in this embodiment. Control signals 214 indicative of the operation of the motor 220 may also be communicated from the motor 220 to the control unit 26 to complete a feedback control loop.

FIG. 8B illustrates another exemplary embodiment of the control unit 26 in the respiratory therapy apparatus 10. In this exemplary embodiment, the control unit 26 receives signals 212 indicative of the user's breathing parameters 88 from the sensor 210. The control unit 26 then utilizes these signals 212 to formulate a control signal 214 which is communicated to the valve 230 within the flow generator 20 to control the pressure 106 delivered to the user. The control signal 214, for example, may alter the position of the valve 230 in order to increase the pressure 106 by pressure step 100. The form of the pressure step 100 and the timing of the pressure step 100 in relation to the breath 90 would be determined by the timing of the altering of the position of the valve 230 as controlled by the control unit 26. Control signals 214 indicative of the operation of the valve 230 may be communicated from the valve 230 to the control unit 26 to complete a feedback control loop.

Methods may include detecting the user's breathing parameters 88 using one or more sensors 210. In some methods, the breathing parameters 88 may indicate the user's progress from wakefulness 124 toward the sleep state 128. The methods may include delivering the pressure 106 to the user at a sub-therapeutic pressure p_{B} and increasing the pressure 106 delivered to the user from the sub-therapeutic pressure p_{B} to the therapeutic pressure p_{T} in one or more pressure steps 100 based upon the user's breathing parameters 88. The steps of basing the pressure steps 100 on demand may also be included in the methods. In some methods, the demand may be determined from the user's breathing parameters 88. In some methods, the demand may be determined from the breath interval 92. In some methods, the demand may be determined from the user's progresses toward the sleep state 128 as determined from the user's breathing parameters 88. The methods may include increasing the pressure 106 by the pressure step 100 during a single breath interval 92. The methods may include the pressure step 100 coinciding with the inhalation 95 portion of the breath 90. The methods may include the pressure step 100 coinciding with the inhalation peak 96. Some methods may include providing the pressure step 100 every other breath 90, every third breath 90, and so forth, and combinations thereof. The methods may also include detecting an apneic event and increasing the pressure 106 to the therapeutic pressure p_{T} by the pressure step 100 during the breath interval 92.

The foregoing discussion discloses and describes merely exemplary embodiments. Upon review of the specification, one of ordinary skill in the art will readily recognize from such discussion, and from the accompanying figures and claims, that various changes, modifications and variations can be made therein without departing from the spirit and scope of the invention as defined in the following claims.

We claim:

1. A method, comprising:
initiating a pressure delivered to a user at a sub-therapeutic pressure;
sensing one or more breathing parameters;
calculating at least two pressure steps from the one or more breathing parameters; and
increasing the pressure by at least two pressure steps while the user is in a state of wakefulness.

2. The method, as in claim 1, further comprising increasing the pressure from the sub-therapeutic pressure to a therapeutic pressure over a rise time, the rise time including at least 10 breath intervals.

3. The method, as in claim 1, further comprising increasing the pressure from the sub-therapeutic pressure to a constant therapeutic pressure.

4. The method, as in claim 1, further comprising increasing the pressure from the sub-therapeutic pressure to a therapeutic pressure over a rise time, the rise time being at least 1 minute.

5. The method, as in claim 1, further comprising:
increasing the pressure from the sub-therapeutic pressure to a constant therapeutic pressure; and
administering a positive airway pressure therapy generally about the therapeutic pressure during at least a portion of a sleep state of the user.

6. The method, as in claim 1, wherein the breathing parameters are indicating normal breathing.

7. The method, as in claim 1, further comprising:
determining a plurality of breath intervals; and
including at least one pressure step initiated and terminated within the breath interval in at least two of the breath intervals of the plurality of breath intervals.

8. The method, as in claim 7, wherein each pressure step is based on the breath interval of a previous breath.

9. The method, as in claim 8, wherein each pressure step is based on the breath interval of the previous breath by a power function.

10. The method, as in claim 9, wherein an exponent m of the power function lies generally in the range −5≦m≦5.

11. The method, as in claim 1, wherein the one or more breathing parameters are selected from the group consisting of the breath interval, breath airflow rate, breath airflow amplitude, mean breath airflow amplitude, inhalation peak, exhalation peak, breath volume, tidal volume, and minute ventilation.

12. The method, as in claim 1, further comprising choosing the pressure step such that the pressure increases from the base pressure to the therapeutic pressure generally over a selected therapeutic pressure delivery time.

13. The method, as in claim 1, further comprising the one or more breathing parameters indicating at least wakefulness and a sleep state.

14. The method, as in claim 13, wherein the one or more breathing parameters are selected from the group consisting of the breath interval, breath airflow rate, breath airflow amplitude, mean breath airflow amplitude, inhalation peak, exhalation peak, breath volume, tidal volume, and minute ventilation.

15. The method, as in claim 13, further comprising:
selecting the pressure step to maintain the pressure generally proximate the sub-therapeutic pressure during wakefulness; and
selecting the pressure step to advance the pressure to the therapeutic pressure as the user attains the sleep state.

16. A method of initiating a positive airway pressure therapy, comprising:
delivering pressurized air at a sub-therapeutic pressure to a user while the user is in a state of wakefulness;
determining one or more breathing parameters of the user;
calculating a pressure step from at least one of the one or more breathing parameters of at least one breath;
increasing the pressure of the pressurized air from the sub-therapeutic pressure to a therapeutic pressure in a plurality of pressure steps;
reaching the therapeutic pressure while the user is in a sleep state; and
administering a positive airway pressure therapy at the therapeutic pressure during the sleep state of the user.

17. A method of initiating a positive airway pressure therapy; comprising:
delivering pressurized air at a sub-therapeutic pressure to a user while the user is in a state of wakefulness;
determining one or more breathing parameters of the user;
determining the transition of the user from the state of wakefulness to the sleep state using the one or more breathing parameters;
increasing the pressure of the pressurized air from the sub-therapeutic pressure to a therapeutic pressure in a plurality of pressure steps;
choosing the pressure steps based on the progression of the user from the state of wakefulness to the sleep state;
reaching the therapeutic pressure while the user is in a sleep state; and
administering a positive airway pressure therapy at the therapeutic pressure during the sleep state of the user.

18. A method, comprising:
initiating a pressure delivered to a user at a sub-therapeutic pressure;
sensing one or more breathing parameters;
calculating at least two pressure steps from the one or more breathing parameters; and
increasing the pressure by at least two pressure steps when the breathing parameters are indicating normal breathing.

19. The method, as in claim 18, further comprising increasing the pressure from the sub-therapeutic pressure to a therapeutic pressure over a rise time, the rise time including at least 10 breath intervals.

20. The method, as in claim 18, further comprising increasing the pressure from the sub-therapeutic pressure to a therapeutic pressure over a rise time, the rise time being at least 1 minute.

21. The method, as in claim 18, further comprising:
increasing the pressure from the sub-therapeutic pressure to a constant therapeutic pressure; and
administering a positive airway pressure therapy generally about the therapeutic pressure during at least a portion of a sleep state of the user.

22. A method, comprising:
initiating a pressure delivered to a user at a sub-therapeutic pressure;
sensing one or more breathing parameters;
determining the transition of the user from the state of wakefulness to the sleep state using the one or more breathing parameters; and
increasing the pressure by a plurality of pressure steps from the sub-therapeutic pressure to the therapeutic pressure while the user is in a state of sleep and the breathing parameters are indicating no apnea.

23. The method, as in claim 22, comprising:
the breathing parameters comprise a least the breath interval;
delivering each pressure step in the plurality of pressure steps over a time interval less than the breath interval; and
including at least one pressure step in at least two of the breath intervals in a plurality of breath intervals.
